SEQUENCE LISTING (11 sequences)

SEQ ID NO: 1 - protein, 10 aa, Homo sapiens
EPKSCDKTHT

SEQ ID NO: 2 - protein, 5 aa, Homo sapiens
CPPCP

SEQ ID NO: 3 - protein, 8 aa, Homo sapiens
APELLGGP

SEQ ID NO: 4 - protein, 12 aa, Homo sapiens
ELKTPLGDTTHT

SEQ ID NO: 5 - protein, 50 aa, Homo sapiens
CPRCPEPKSCDTPPPCPRCPEPKSCDTPPPCPRCPEPKSCDTPPPCPRCP

SEQ ID NO: 6 - protein, 7 aa, Homo sapiens
ESKYGPP

SEQ ID NO: 7 - protein, 5 aa, Homo sapiens
CPSCP

SEQ ID NO: 8 - protein, 8 aa, Homo sapiens
APEFLGGP

SEQ ID NO: 9 - skipped

SEQ ID NO: 10 - protein, 10 aa, Homo sapiens
CCVECPPPCP

SEQ ID NO: 11 - protein, 7 aa, Homo sapiens
APPVAGP

(19) United States
(12) Patent Application Publication
(10) Pub. No.: US 2026/0356942 A1
(43) Pub. Date: Nov. 12, 2026

(54) ANTI-MAST CELL ANTIBODIES

(71) Applicant: argenx BV, Ghent (BE)

(72) Inventors: Laurent L. REBER, Toulouse (FR); William WORRALL, Toulouse (FR)

(21) Appl. No.: 19/539,406

(22) Filed: Feb. 13, 2026

Related U.S. Application Data
(63) Continuation of application No. PCT/EP2024/076659, filed on Sep. 23, 2024.

(30) Foreign Application Priority Data
Sep. 25, 2023 (GB) 2314662.4

(51) Int. Cl. C07K 16/28; A61K 39/00; A61P 37/08

(52) U.S. Cl. CPC C07K 16/2803; A61P 37/08; A61K 2039/505; A61K 2039/54

(57) ABSTRACT

The present invention relates to methods of depleting mast cells. The invention also relates to methods of treating a mast cell disease in a subject in need thereof. The methods involve the administration of an anti-mast cell antibody wherein the antibody is administered via a slow absorption delivery route, in particular wherein the antibody is administered subcutaneously. Administration of anti-mast cell antibodies via a slow absorption delivery route, for example subcutaneously or intradermally, can avoid the anaphylaxis seen when anti-mast cell antibodies are administered via alternative routes e.g. intravenously.

### CROSS REFERENCE TO RELATED APPLICATIONS

This application is a Continuation of International Patent Application No. PCT/EP2024/076659, filed Sep. 23, 2024, which claims the benefit of and priority to GB Application No. 2314662.4, filed Sep. 25, 2023, the contents of each of which are incorporated herein by reference in their entirety.

### SUBMISSION OF SEQUENCE LISTING XML

The content of the following submission of Sequence Listing XML is incorporated herein by reference in its entirety: a computer readable form (CRF) of the Sequence Listing (file name: ARGX-T2212.xml, date created: Feb. 12, 2026, size: 9,590 bytes).

### FIELD OF THE INVENTION

The present invention relates to methods of depleting mast cells. The invention also relates to methods of treating a mast cell disease in a subject in need thereof. The methods involve the administration of an anti-mast cell antibody wherein the antibody is administered via a slow absorption delivery route, in particular wherein the antibody is administered subcutaneously.

### BACKGROUND OF THE INVENTION

Mast cells are inflammatory immune sentinel cells that reside in tissues across the body, particularly at interfaces with the external environment, for example at mucosal surfaces of the gut and lungs, in the skin, and around blood vessels. Mast cells play an important role in many physiological functions such as vasodilation, wound healing, and angiogenesis. Most significantly, they play a central role in the body's adaptive immune response. Given the importance of mast cells in inducing the inflammatory cascade, they have also been implicated in driving numerous allergic, inflammatory, and pruritic disorders when activated. Mast cells may be activated by a number of endogenous and exogenous stimuli including allergen-specific IgE autoantibodies, complement, toll-like receptor (TLR) agonists, alarmins, cytokines, neuropeptides, drugs and venoms. Upon stimulation, mast cells release preformed, granule-stored mediators, such as histamine, tryptase, chymase, and TNF-alpha into the extracellular space in a process termed ‘degranulation’. This is followed by a second wave of de novo synthesis of prostaglandins and leukotrienes, which ultimately results in a rapid inflammatory response. The best studied mechanism by which mast cells become activated is through IgE-mediated cross-linking of the FεRI, which is highly expressed on human mast cells. In addition to FcεRI, human mast cells also express Fc receptors for IgG (FcγRs), receptors for complement proteins, toll-like receptors (TLRs), c-KIT receptors, cytokine receptors, as well as the G-protein coupled receptor MRGPRX2, all of which are involved in mast cell activation and immune response.

Mast cell diseases are characterized by unwanted mast cell degranulation. This may be caused by an increased sensitivity of the patient's mast cells to activation by external stimuli. The most common are the type I hypersensitivity reactions, for example urticaria (hives), which occur as a result of an exaggerated IgE-mediated immune response to an antigen that should not normally trigger the immune system. Another mast cell disease is mastocytosis, a condition characterised by an excess number of mast cells in the body. Mastocytosis is frequently associated with mutations in the c-kit gene encoding the c-KIT receptor, particularly gain-of-function mutations which lead to constitutive c-KIT activation. The abnormally high number of mast cells found in patients with mastocytosis and their potential to release large amounts of histamine into the blood means that patients with the disease are at a much higher risk of severe allergic reactions such as anaphylaxis.

A number of therapies have been proposed to treat mast cell diseases. Therapies, such as the anti-IgE antibody, omalizumab, are designed to inhibit a specific mast cell trigger. Other therapies aim to target mast cell mediators; for example, antihistamines are the first line of treatment in many mast cell diseases. However, evidence suggests that a more comprehensive suppression of mast cell numbers or activity, rather than targeting a single trigger or mediator, would result in broader efficacy. Tyrosine kinase inhibitors (TKIs) that inhibit the c-KIT receptor have also been developed due to the involvement of c-KIT signalling in mast cell differentiation, maturation and survival. However, some of the downsides to TKIs targeting c-KIT include unwanted reactivity with other related kinases and the emergence of TKI-resistant mast cells.

Recently, anti-c-KIT antibodies have been developed to achieve more selective targeting of mast cells. However, mast cells have proved to be a particularly difficult cell type to target with therapeutic antibodies due to the fact that mast cells highly express Fc receptors. This means that they are readily engaged in adaptive immune responses triggered by antibodies themselves. Thus, a significant concern for systemic dosing of c-KIT-targeting antibodies is the potential for mast cell activation and degranulation caused by c-KIT-dependent clustering of mast cell FcγRs via the antibody Fc domain. Accordingly, an Fc-dead anti-c-KIT antibody, barzolvolimab, was engineered and is currently in phase 2 trials for the treatment of chronic spontaneous urticaria (Alvarado et al. 2022; Basic and Translational Allergy Immunology; 77: 2393-2403). Barzolvolimab binds to c-KIT and blocks SCF-dependent signalling, but importantly this antibody does not engage the innate immune system, as it has been engineered to prevent the engagement of FcγRs using key mutations in the Fc portion of the antibody to abolish this function.

### SUMMARY OF INVENTION

As reported herein, anti-mast cell antibodies that are “Fc-dead” have very limited capacity for mast cell depletion. This follows from the absence of Fc effector function, for example the antibody-dependent cellular cytotoxicity mediated by antibody Fc region binding to Fc receptors on immune cells, including mast cells themselves.

The present inventors thus sought to re-introduce Fc effector function into anti-mast cell antibodies so as to enhance mast cell depletion whilst, at the same time, avoiding the unwanted mast cell activation and anaphylaxis that follows from Fc receptor engagement. As demonstrated herein, effective mast cell depletion and a reduction in anaphylaxis can be achieved by optimising the route of administration of anti-mast cell antibodies. More specifically, administration of anti-mast cell antibodies via a slow absorption delivery route, for example subcutaneously or intradermally, can avoid the anaphylaxis seen when anti-mast cell antibodies are administered via alternative routes e.g. intravenously.

By employing a slow absorption delivery route, it is thought that the slow absorption of the anti-mast cell antibody into the patient's bodily fluids, either lymph fluid or blood, will bring about the relatively slow depletion of mast cells. Without wishing to be bound by theory, it is thought that the slow rate of depletion results in a slower release of histamines and other chemical mediators following lysis of the mast cells. This, in turn, reduces the risk for patients with mast cell diseases of suffering severe allergic reactions such as anaphylaxis.

In a first aspect, the present invention provides a method of depleting mast cells in a subject, the method comprising administering an effective amount of an anti-mast cell antibody to the subject, wherein the anti-mast cell antibody is administered to the subject via a slow absorption delivery route.

In a second aspect, the present invention provides a method of treating a mast cell disease in a subject in need thereof, the method comprising administering a therapeutically effective amount of an anti-mast cell antibody to the subject, wherein the anti-mast cell antibody is administered to the subject via a slow absorption delivery route.

In certain embodiments, the anti-mast cell antibody is administered subcutaneously.

In certain embodiments, the anti-mast cell antibody is administered via a subcutaneous implant.

In certain embodiments, the anti-mast cell antibody is administered intradermally.

In certain embodiments, the anti-mast cell antibody is not administered intravenously.

In certain embodiments, the anti-mast cell antibody specifically binds to a protein expressed on the surface of mast cells. In certain embodiments, the anti-mast cell antibody is an anti-c-KIT antibody. In certain preferred embodiments, the anti-mast cell antibody comprises an Fc region. In certain embodiments, the Fc region possesses one or more Fc effector functions. In certain embodiments, the Fc region possesses one or more enhanced Fc effector functions relative to a wild-type Fc region. In certain embodiments, the one or more Fc effector functions are selected from: antibody-dependent cellular cytotoxicity (ADCC), antibody-dependent phagocytosis (ADCP), and complement dependent cytotoxicity (CDC). In certain embodiments, the Fc region possesses the Fc effector function antibody-dependent cellular cytotoxicity (ADCC). In certain embodiments, the Fc region possesses enhanced Fc effector function antibody-dependent cellular cytotoxicity (ADCC) relative to a wild-type Fc region.

In certain embodiments, the anti-mast cell antibody is a human IgG antibody. In certain embodiments, the Fc region comprises the amino acid substitutions S239D/I332E. In certain embodiments, the Fc region is afucosylated. In certain embodiments, the Fc region is galactosylated.

In certain embodiments, the mast cell disease is mastocytosis. In certain embodiments, the subject has an abnormally high mast cell number. In certain embodiments, the subject is a human.

In certain embodiments, the anti-mast cell antibody is formulated in a pharmaceutical composition. In certain embodiments, the pharmaceutical composition is formulated for slow absorption of the anti-mast cell antibody, optionally wherein the pharmaceutical composition is a modified-release formulation selected from: slow-release, extended-release, prolonged-release, sustained-release, delayed-release, or controlled-release.

In certain embodiments, the method reduces or prevents mast cell activation or mast cell degranulation. In certain embodiments, the method reduces or prevents allergic reactions or anaphylaxis. In certain embodiments, administration of the anti-mast cell antibody does not trigger mast cell degranulation, an allergic reaction and/or anaphylaxis.

In a third aspect, the present invention provides an anti-mast cell antibody for use in a method of treating a mast cell disease, wherein the method comprises administering a therapeutically effective amount of an anti-mast cell antibody to the subject, wherein the anti-mast cell antibody is administered to the subject via a slow absorption delivery route.

In a fourth aspect, the present invention provides use of an anti-mast cell antibody in the manufacture of a medicament for treating a mast cell disease, wherein the treating comprises administering a therapeutically effective amount of an anti-mast cell antibody to a subject, wherein the anti-mast cell antibody is administered to the subject via a slow absorption delivery route.

### BRIEF DESCRIPTION OF THE DRAWINGS

FIG. 1A-FIG. 1C shows the results of a desensitization experiment observed in wild-type mice administered an intraperitoneal injection of an anti-c-KIT antibody. (FIG. 1A) Mice were administered increasing doses (0.1, 0.3, 1.0, 3.0 and 10 μg/injection) of an anti-c-KIT antibody having enhanced ADCC activity (ADCC+ anti-CD117), an ADCC+ isotype control or a PBS control every 30 minutes, and the extent of anaphylaxis (i.e. a decrease in body temperature) was measured. (FIG. 1B) After the desensitization phase, the same mice were challenged with a larger bolus of anti-c-KIT antibody on day 0, day 2, and again on day 4, and body temperature was measured. (FIG. 1C) On day 7, mast cells in the peritoneal cavity were quantified.

FIG. 2A-FIG. 2D shows the extent of anaphylactic reaction and the release of mast cell proteases (MCPT-6 and MCPT-1) in mice receiving a 30 μg injection of an anti-c-KIT ADCC+ antibody or an anti-c-KIT Fc-dead antibody administered intraperitoneally (FIG. 2B), intravenously (FIG. 2C), or subcutaneously (FIG. 2D). One hour after the challenge with the antibodies, the mice were euthanised, blood samples were taken and serum isolated to quantify the release of mast cell-specific proteases, as depicted in (FIG. 2A).

FIG. 3A-FIG. 3C shows that intraperitoneal injection of an anti-c-KIT ADCC+ antibody or an anti-c-KIT wildtype antibody depletes mast cells in the peritoneal cavity (FIG. 3B), but triggers anaphylaxis (FIG. 3C). As depicted in (FIG. 3A), mast cell numbers were evaluated on day 7 following i.p administration of the antibodies.

FIG. 4A-FIG. 4F shows the effect of three doses of anti-c-KIT antibodies (Fc-dead, WT and ADCC+) administered subcutaneously on days 0, 2 and 4 on anaphylactic reaction and mast cell depletion (FIG. 4A). As shown in (FIG. 4B), subcutaneous administration of anti-c-KIT antibodies does not trigger anaphylaxis. On day 7 following administration, mast cell numbers were quantified in the peritoneal cavity (FIG. 4C), back skin (FIG. 4D), ear skin (FIG. 4E), and mesentery (FIG. 4F).

FIG. 5A-FIG. 5B shows the effect of different doses of anti-c-KIT ADCC+ antibody on mast cell numbers. (FIG. 5A) Mice received a subcutaneous dose of either 1, 5, 10 or 30 μg antibody on day 0 and again on day 7, before they were euthanised on day 14. (FIG. 5B) Mast cell numbers in the peritoneal cavity were quantified.

FIG. 6A-FIG. 6D shows the efficacy of anti-c-KIT antibodies (Fc-dead, WT and ADCC+) on mast cell number in different organs. (FIG. 6A) Mice received a subcutaneous dose of 30 μg antibody on day 0 and again on day 7, before they were euthanised on day 14. (FIG. 6B) and (FIG. 6C) show the mast cell profile in the peritoneal cavity. (FIG. 6D) shows the numbers of mast cells in the mesentery, back skin, ear skin, lung, small intestine and large intestine.

FIG. 7A-FIG. 7H shows the effects of anti-c-KIT antibodies (Fc-dead, WT and ADCC+) on different leukocyte populations.

FIG. 8 shows the effect of anti-c-KIT antibodies (Fc-dead, WT and ADCC+) on bone marrow progenitor populations.

FIG. 9 shows the effect of antibody-induced mast cell depletion in reducing anaphylaxis severity in an IgE passive systemic anaphylaxis model. Mice received three subcutaneous injections of an anti-c-KIT antibody (either Fc-dead, WT or ADCC+) on day 0, day 2 and day 4, before IgE sensitisation on day 7 and antigen challenge on day 8. Core body temperature was monitored for an hour after the challenge to examine hypothermia.

FIG. 10A-FIG. 10C shows the effect of antibody-induced mast cell depletion in reducing anaphylaxis severity in an IgE passive systemic anaphylaxis model. (FIG. 10A) Mice received two subcutaneous injections of an anti-c-KIT antibody (either Fc-dead, WT or ADCC+) on day 0 and day 7, before IgE sensitisation on day 14 and antigen challenge on day 15. (FIG. 10B) Core body temperature was monitored for an hour after the challenge to examine hypothermia. (FIG. 10C) 60 minutes post-challenge, blood was harvested for serum analysis of mast cell specific proteases, MCPT-1 and MCPT-6, which were later quantified by ELISA.

FIG. 11A-FIG. 11C shows the effect of anti-c-KIT antibody (Fc-dead and ADCC+) treatments on mrgprb2-mediated anaphylaxis. (FIG. 11A) Mice received two subcutaneous injections of an anti-c-KIT antibody on day 0 and day 7, before ciprofloxacin challenge on day 14. (FIG. 11B) Core body temperature was monitored for an hour after the challenge to examine hypothermia. (FIG. 11C) 60 minutes post-challenge, blood was harvested for serum analysis of mast cell specific proteases, MCPT-1 and MCPT-6, which were later quantified by ELISA.

### DETAILED DESCRIPTION

### A. Definitions

Unless defined otherwise, all technical and scientific terms used herein have the same meaning as commonly understood by the ordinary person skilled in the art to which the invention pertains. Without limiting any term, further clarifications of some of the terms used herein are provided below.

As used herein, the term “immunoglobulin” includes a polypeptide having a combination of two heavy and two light chains whether or not it possesses any relevant specific immunoreactivity. “Antibodies” refer to such assemblies which have significant known specific immunoreactive activity to an antigen of interest. Antibodies and immunoglobulins comprise light and heavy chains, with or without an interchain covalent linkage between them. Basic immunoglobulin structures in vertebrate systems are relatively well understood.

The generic term “immunoglobulin” comprises five distinct classes of antibody that can be distinguished biochemically. With regard to IgG, immunoglobulins comprise two identical light polypeptide chains of molecular weight approximately 23,000 Daltons, and two identical heavy chains of molecular weight 53,000-70,000 Daltons. The four chains are joined by disulfide bonds in a “Y” configuration wherein the light chains bracket the heavy chains starting at the mouth of the “Y” and continuing through the variable region. The light chains of an antibody are classified as either kappa or lambda (κ,λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA, IgD or IgE, respectively. The immunoglobulin subclasses (isotypes) e.g., IgG1, IgG2, IgG3, IgG4, IgA1, etc. are well characterised and are known to confer functional specialization. Modified versions of each of these classes and isotypes are readily discernible to the skilled artisan in view of the instant disclosure and, accordingly, are within the scope of the instant invention.

The variable region of an antibody allows the antibody to selectively recognize and specifically bind epitopes on antigens. That is, the variable light (VL) domain and variable heavy (VH) domain of an antibody combine to form the variable region that defines a three-dimensional antigen binding site. This quaternary antibody structure forms the antigen binding site present at the end of each arm of the Y. More specifically, the antigen binding site is defined by three complementary determining regions (CDRs) on each of the VH and VL chains.

The terms “variable region” and “variable domain” are used herein interchangeably and are intended to have equivalent meaning. The term “variable” refers to the fact that certain portions of the variable domains VH and VL differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the VL domain and the VH domain which form part of the antigen binding site. The first, second and third hypervariable loops of the VLambda light chain domain are referred to herein as L1(λ), L2(λ) and L3(λ) and may be defined as comprising residues 24-33 (L1(λ), consisting of 9, 10 or 11 amino acid residues), 49-53 (L2(λ), consisting of 3 residues) and 90-96 (L3(λ), consisting of 5 residues) in the VL domain (Morea et al., Methods 20:267-279 (2000)). The first, second and third hypervariable loops of the VKappa light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the VL domain (Morea et al., Methods 20:267-279 (2000)). The first, second and third hypervariable loops of the VH domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the VH domain (Morea et al., Methods 20:267-279 (2000)).

Unless otherwise indicated, the terms L1, L2 and L3 respectively refer to the first, second and third hypervariable loops of a VL domain, and encompass hypervariable loops obtained from both Vkappa and Vlambda isotypes. The terms H1, H2 and H3 respectively refer to the first, second and third hypervariable loops of the VH domain, and encompass hypervariable loops obtained from any of the known heavy chain isotypes, including γ, ε, δ, α or μ.

The hypervariable loops L1, L2, L3, H1, H2 and H3 may each comprise part of a “complementarity determining region” or “CDR”, as defined below. The terms “hypervariable loop” and “complementarity determining region” are not strictly synonymous, since the hypervariable loops (HVs) are defined on the basis of structure, whereas complementarity determining regions (CDRs) are defined based on sequence variability (Kabat et al., Sequences of Proteins of Immunological Interest, 5th Ed. Public Health Service, National Institutes of Health, Bethesda, MD., 1983) and the limits of the HVs and the CDRs may be different in some VH and VL domains.

The CDRs of the VL and VH domains can typically be defined as comprising the following amino acids: residues 24-34 (LCDR1), 50-56 (LCDR2) and 89-97 (LCDR3) in the light chain variable domain, and residues 31-35 or 31-35b (HCDR1), 50-65 (HCDR2) and 95-102 (HCDR3) in the heavy chain variable domain; (Kabat et al., Sequences of Proteins of Immunological Interest, 5th Ed. Public Health Service, National Institutes of Health, Bethesda, MD. (1991)). Thus, the HVs may be comprised within the corresponding CDRs and references herein to the “hypervariable loops” of VH and VL domains should be interpreted as also encompassing the corresponding CDRs, and vice versa, unless otherwise indicated.

The more highly conserved portions of variable domains are called the framework region (FR), as defined below. The variable domains of native heavy and light chains each comprise four FRs (FR1, FR2, FR3 and FR4, respectively), largely adopting a β-sheet configuration, connected by the three hypervariable loops. The hypervariable loops in each chain are held together in close proximity by the FRs and, with the hypervariable loops from the other chain, contribute to the formation of the antigen binding site of antibodies. Structural analysis of antibodies revealed the relationship between the sequence and the shape of the binding site formed by the complementarity determining regions (Chothia et al., J. Mol. Biol. 227: 799-817 (1992)); Tramontano et al., J. Mol. Biol, 215:175-182 (1990)). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

As used herein, the term “CDR” or “complementarity determining region” means the non-contiguous antigen binding sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

TABLE 2
CDR definitions

CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the term “constant region” refers to the portion of the antibody outside of the variable domains or variable regions. Immunoglobulin light chains have a single domain “constant region”, typically referred to as the “CL or CL1 domain”. This domain lies C terminal to the VL domain. Immunoglobulin heavy chains differ in their constant region depending on the class of immunoglobulin ((γ, μ, α, δ, ε). Heavy chains γ, a and δ have a constant region consisting of three immunoglobulin domains (referred to as CH1, CH2 and CH3) with a flexible hinge region separating the CH1 and CH2 domains. Heavy chains μ and ε have a constant region consisting of four domains (CHT-CH4). The constant domains of the heavy chain are positioned C terminal to the VH domain.

The numbering of the amino acids in the heavy and light immunoglobulin chains run from the N-terminus at the forked ends of the Y configuration to the C-terminus at the bottom of each chain. Different numbering schemes are used to define the constant domains of the immunoglobulin heavy and light chains. In accordance with the EU numbering scheme, the heavy chain constant domains of an IgG molecule are identified as follows: CH1—amino acid residues 118-215; CH2—amino acid residues 231-340; CH3—amino acid residues 341-446. In accordance with the Kabat numbering scheme, the heavy chain constant domains of an IgG molecule are identified as follows: CH1—amino acid residues 114-223; CH2—amino acid residues 244-360; CH3—amino acid residues 361-477.

The “Fc domain” or “Fc region” typically defines the portion of the constant region of a heavy chain including the CH2 and CH3 domains. The Fc region may also include some residues from the hinge region. The “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux K. H. et al. J. Immunol. 161:4083-90 1998). Antibodies of the invention comprising a “fully human” hinge region may contain one of the hinge region sequences shown in Table 3 below.

TABLE 3
Human hinge sequences

| IgG | Upper hinge | Middle hinge | Lower hinge |
|---|---|---|---|
| IgG1 | EPKSCDKTHT | CPPCP | APELLGGP |
| | (SEQ ID NO: 1) | (SEQ ID NO: 2) | (SEQ ID NO: 3) |
| IgG3 | ELKTPLGDTTHT | CPRCP(EPKSCDTPPPCPRCP)₃ | APELLGGP |
| | (SEQ ID NO: 4) | (SEQ ID NO: 5) | (SEQ ID NO: 3) |
| IgG4 | ESKYGPP | CPSCP | APEFLGGP |
| | (SEQ ID NO: 6) | (SEQ ID NO: 7) | (SEQ ID NO: 8) |
| IgG2 | ERK | CCVECPPPCP | APPVAGP |
| | (SEQ ID NO: 9) | (SEQ ID NO: 10) | (SEQ ID NO: 11) |

The Fc region interacts with cell surface receptors called Fc receptors and some proteins of the complement system. This region allows antibodies to activate the immune system, for example, through binding to Fc receptors, and is therefore responsible for mediating antibody effector functions, which are defined below.

“Antibody effector functions” are an important part of the humoral immune response and form an essential link between innate and adaptive immunity. Most of these effector functions are induced via the constant (Fc) region of the antibody, which can interact with complement proteins and specialized Fc receptors. The term “antibody effector function” is used herein interchangeably with “Fc effector function” or “Fc region effector function”. The most well-known Fc-mediated antibody effector functions are antibody-dependent cell-mediated cytotoxicity (ADCC), antibody-dependent cellular phagocytosis (ADCP), and complement-dependent cytotoxicity (CDC). In ADCC, FcγRs on the surface of effector cells (natural killer cells, macrophages, monocytes and eosinophils) bind to the Fc region of an IgG which itself is bound to a target cell. This interaction induces the release of cytotoxic granules (containing perforins and granzymes) from the effector cell resulting in lysis and ultimately killing of the target cell.

“Fragment”—the term “fragment”, as used in the context of antibodies of the invention, refers to a part or portion of an antibody or antibody chain comprising fewer amino acid residues than an intact or complete antibody or antibody chain. The term “antigen binding fragment” refers to a polypeptide fragment of an immunoglobulin or antibody that binds antigen or competes with intact antibody (i.e., with the intact antibody from which they were derived) for antigen binding. As used herein, the term “fragment” of an antibody molecule includes antigen binding fragments of antibodies, for example, an antibody light chain variable domain (VL), an antibody heavy chain variable domain (VH), a single chain antibody (scFv), a F(ab′)2 fragment, a Fab fragment, an Fd fragment, an Fv fragment, a one-armed (monovalent) antibody, diabodies, triabodies, tetrabodies or any antigen binding molecule formed by combination, assembly or conjugation of such antigen binding fragments. The term “antigen binding fragment” as used herein is further intended to encompass antibody fragments selected from the group consisting of unibodies, domain antibodies and nanobodies. Fragments can be obtained, e.g., via chemical or enzymatic treatment of an intact or complete antibody or antibody chain or by recombinant means.

As used herein, the term “modified-release formulation” is used to refer to a conventional, immediate-release formulation that has been modified to alter the timing and/or rate of release of the active ingredient from the formulation when administered by the same route. The term may encompass various different modified forms including, but not limited to, slow-release formulations, extended-release formulations, sustained-release formulations, delayed-release formulations, controlled-release formulations, long-acting formulations, or subcutaneous depot formulations. Extended-release drug products for instance allow for a reduction in dosage frequency as compared to that drug presented as an immediate-release dosage form. Delayed-release drug products may release a discrete portion or portions of drug at a time other than promptly after administration. Modified-release formulations and strategies for altering the timing and/or rate of release of the active ingredient from the formulation are known to the skilled person. One popular strategy to develop long-acting systems is to encapsulate the original drug in a hydrogel or macro-sized particles, which can be injected or implanted subcutaneously to release the payloads via gradual degradation. An alternative method is to coat the surface of subcutaneous depots with a masking layer that can provide a hydrophilic interface between the tissue fluids and depots, significantly reducing protein adsorption and cell attachment. There are also a number of sustained-release lipid-based platforms for example liposomal systems whereby release of the liposome-encapsulated drug is dependent on the diffusion of the drug across the lipid bilayer (Rahnfeld & Luciani, 2020; Pharmaceutics; 12(6): 567).

As used herein, the term “mast cell disease” or “mast cell disorder” refers to a group of diseases in which mast cell dysfunction plays the primary role in the pathogenesis of the disease. Mast cell diseases are conditions in which mast cells are either increased in number, hyper-reactive, or both. Symptoms are therefore caused by the proliferation and inappropriate activation of excess mast cells and/or the inappropriate release of mast cell mediators from hypersensitive mast cells. Examples of mast cell diseases include mastocytosis. The symptoms experienced by patients with mast cell disease are caused by the excessive release of mast cell mediators, most notably histamine. The symptoms experienced depend on the affected organ, e.g. wheals, redness and itching in the skin, or cramps and diarrhoea in the gastrointestinal tract. Sometimes patients will present with an isolated finding, such as the rash of cutaneous mastocytosis; other patients will present with a broad constellation of symptoms, e.g. flushing, pre-syncope, diarrhoea and cramps.

As used herein, the term “mastocytosis” refers to a mast cell disease, typically caused by clonal proliferation of mast cells, with an abnormal accumulation of these cells in tissues including the skin, bone marrow and gastrointestinal tract.

As used herein, the term “slow absorption delivery route” refers to a route of drug delivery that results in the slow absorption of the active ingredient from the site of injection into the patient's bodily fluid, either lymph fluid or blood. Intravenous administration is the most rapid route of administration since the active ingredient is injected directly into the blood stream with no requirement for absorption. The muscles are also richly supplied with blood vessels and therefore intramuscular administration results in rapid absorption of the active ingredient through the muscle tissue and into the blood stream. As used herein, the term “slow absorption delivery route” may be “slow” relative to intravenous administration and/or relative to intramuscular administration. Subcutaneous tissue has a relatively low density of blood vessels and therefore subcutaneous administration is generally considered by practitioners to represent a “slow absorption delivery route” since the active ingredient diffuses through the tissue at a slow, sustained rate. Intradermal administration is classified as another “slow absorption delivery route”. Intradermal injections are administered into the dermis, just below the epidermis, and have the longest absorption time of all parental routes since this tissue layer has a limited blood supply.

As used herein, the terms “treat”, “treating” and “treatment” refer to the therapeutic treatment of a mast cell disease in a subject. The methods of treatment described herein involve administration of an anti-mast cell antibody to a subject via a slow absorption delivery route. The treatment results in ameliorating or stabilising the disease, reducing or eliminating the symptoms of the disease, or slowing or eliminating the progression of the disease. The treatment may also include prophylactic treatment of the disease.

As used herein, the term “therapeutically effective amount” refers to the quantity of the anti-mast cell antibody which will elicit the desired biological response (depletion of mast cells) in the subject. The amount deemed to be an effective amount may vary depending on the anti-mast cell antibody, the stage and/or severity of the disease, or the age and/or weight of the subject to be treated. A therapeutically effective amount can readily be determined by one skilled in the art, for example a physician treating the subject. A therapeutically effective amount is typically an amount of anti-mast cell antibody effective to ameliorate or reduce one or more symptoms of the mast cell disease.

As used herein, the term “subject” refers to a mammal, preferably a human. The subject may be either a male or a female. The subject may exhibit one or more symptoms consistent with a mast cell disease. In certain embodiments, the subject may be a patient, where a patient is an individual who is under medical care and/or actively seeking medical care for treatment of a mast cell disease.

As used herein, the term “anaphylaxis” or “anaphylactic shock” refers to a severe, potentially life-threatening allergic reaction. Anaphylaxis usually develops within minutes of contact with an allergen, but sometimes the reaction can happen up to 4 hours later. Signs of anaphylaxis include: itchy skin or a raised, red skin rash; swollen eyes, lips, hands and feet; feeling lightheaded or faint; swelling of the mouth, throat or tongue, which can cause breathing and swallowing difficulties; wheezing; abdominal pain, nausea and vomiting; and collapse and unconsciousness. Anaphylaxis occurs rapidly and systemically, affecting one or more organ systems, generally where mast cells reside in relative abundance. Underlying the pathophysiology of anaphylaxis is exposure to allergen or other factors that activate mast cells or basophils, prompting degranulation and immediate (5-30 minutes) release of preformed mediators (histamine, tryptase, carboxypeptidase A, and proteoglycans), synthesis of arachidonic acid metabolites (prostaglandins, leukotrienes), and platelet-activating factor (PAF), and delayed-phase (2-6 hours) generation of cytokines (TNF-α) and chemokines resulting from increased gene expression. These mediators are thought to be responsible for the symptoms associated with anaphylaxis. The models that are most widely used to study anaphylactic responses are passive cutaneous anaphylaxis (PCA) and passive systemic anaphylaxis (PSA), which can be examined in genetically modified murine strains.

### B. Methods of Depleting Mast Cells & Methods of Treating Mast Cell Diseases

In a first aspect, the present invention is directed to methods of depleting mast cells in a subject. A number of pathological conditions are characterized by either the presence of abnormal mast cells or by the accumulation of an excessive number of mast cells. Accordingly, it is an object of the present invention to deplete mast cell numbers in a subject in need thereof.

In a further aspect, the present invention provides methods of treating a mast cell disease in a subject in need thereof. The subject in need thereof will typically be characterised by the presence of abnormal mast cells and/or by the accumulation of an excessive number of mast cells.

The methods of the present invention comprise the administration of an effective amount of an anti-mast cell antibody to the subject, wherein the anti-mast cell antibody is administered via a slow absorption delivery route. As reported elsewhere herein, the administration of anti-mast cell antibodies via a slow absorption delivery route allows for the effective depletion of mast cells whilst avoiding the anaphylaxis that can occur following anti-mast cell antibody administration via alternative routes e.g. intravenous administration.

In certain embodiments, the methods deplete total mast cell numbers in the subject. In certain embodiments, the methods provided herein deplete mast cells in one or more of the following tissues or organs: skin; mesentery; lung; small intestine; and large intestine. As used herein, the term “depleting mast cells” means that the number of mast cells present in the subject after administration of the anti-mast cell antibody is reduced compared to the number of mast cells present in the subject prior to administration of the anti-mast cell antibody. In certain embodiments, “depleting mast cells” means that the number of mast cells present in the subject after administration of anti-mast cell antibody is reduced below a pre-determined threshold.

The skilled person is aware of appropriate means of assessing the number of mast cells in a subject. The number of mast cells may be determined by histochemical methods. For example, a tissue sample from the subject may be biopsied, fixed in formalin and paraffin-embedded. Sections may then be stained with toluidine blue, methylene blue or Csaba stain which bind to heparin and other acidic mucopolysaccharides contained in mast cells and stain them metachromatically. Alternatively, the total mast cell number may be estimated based on the levels of serum plasma tryptase, a marker of mast cell burden. Mast cells release tryptase in response to external stimuli, but they also release small amounts of tryptase in the steady state resulting in circulating tryptase levels that correlate to the number of mast cells in tissues.

### Routes of Administration

As demonstrated in the examples herein, mast cells can be safely and effectively depleted by the administration of an anti-mast cell antibody according to an optimised delivery route. In particular, the methods of the invention require that the anti-mast cell antibody is administered to the subject via a slow absorption delivery route. The term “slow absorption delivery route” is defined elsewhere herein and typically refers to administration of a drug via a route that is slower than intravenous administration i.e. wherein the drug is administered directly into the circulation. Delivery routes that are recognised in the art as “slow absorption delivery routes” include but are not limited to subcutaneous administration and intradermal administration. These routes of administration involve delivery into tissues that have a limited blood supply and therefore the drug must diffuse from the injection site through the tissue to reach the blood stream. This results in a “slow absorption delivery route”, for example as compared with intravenous administration.

Accordingly, in embodiments of the first and second aspects of the invention, the anti-mast cell antibody may be administered via any delivery route known to the skilled person to effect slow absorption of the antibody. In certain embodiments, the anti-mast cell antibody is administered subcutaneously. In certain embodiments, the anti-mast cell antibody is administered via a subcutaneous implant. In certain embodiments, the anti-mast cell antibody is administered intradermally. For the same reasoning, in all embodiments of the first and second aspects of the invention, the anti-mast cell antibody is not administered to the subject via a delivery route that is known to the skilled person to effect rapid and immediate targeting of the mast cells. In certain embodiments, the anti-mast cell antibody is not administered intravenously.

Provided herein is a method of depleting mast cells in a subject, the method comprising administering an effective amount of an anti-mast cell antibody to the subject, wherein the anti-mast cell antibody is administered to the subject subcutaneously.

Provided herein is a method of depleting mast cells in a subject, the method comprising administering an effective amount of an anti-mast cell antibody to the subject, wherein the anti-mast cell antibody is administered to the subject via a subcutaneous implant.

Provided herein is a method of depleting mast cells in a subject, the method comprising administering an effective amount of an anti-mast cell antibody to the subject, wherein the anti-mast cell antibody is administered to the subject intradermally.

Provided herein is a method of depleting mast cells in a subject, the method comprising administering an effective amount of an anti-mast cell antibody to the subject, wherein the anti-mast cell antibody is not administered intravenously.

Provided herein is a method of depleting mast cells in a subject, the method comprising administering an effective amount of an anti-mast cell antibody to the subject, wherein the anti-mast cell antibody is administered to the subject via a slow absorption delivery route, and wherein the anti-mast cell antibody is not administered intravenously.

Provided herein is a method of treating a mast cell disease in a subject in need thereof, the method comprising administering a therapeutically effective amount of an anti-mast cell antibody to the subject, wherein the anti-mast cell antibody is administered to the subject subcutaneously.

Provided herein is a method of treating a mast cell disease in a subject in need thereof, the method comprising administering a therapeutically effective amount of an anti-mast cell antibody to the subject, wherein the anti-mast cell antibody is administered to the subject via a subcutaneous implant.

Provided herein is a method of treating a mast cell disease in a subject in need thereof, the method comprising administering a therapeutically effective amount of an anti-mast cell antibody to the subject, wherein the anti-mast cell antibody is administered to the subject intradermally.

Provided herein is a method of treating a mast cell disease in a subject in need thereof, the method comprising administering a therapeutically effective amount of an anti-mast cell antibody to the subject, wherein the anti-mast cell antibody is not administered to the subject intravenously.

Provided herein is a method of treating a mast cell disease in a subject in need thereof, the method comprising administering a therapeutically effective amount of an anti-mast cell antibody to the subject, wherein the anti-mast cell antibody is administered to the subject via a slow absorption delivery route, and wherein the anti-mast cell antibody is not administered intravenously.

Tₘₐₓ is the time it takes to reach maximum drug concentration (Cₘₐₓ) in the plasma after administration of said drug. In general, the slow absorption delivery route will result in an increased Tₘₐₓ of the anti-mast cell antibody compared to administration via a known rapid delivery route such as intravenous administration. In certain embodiments, the anti-mast cell antibody is administered to the subject via a slow absorption delivery route such that the Tₘₐₓ of the anti-mast cell antibody is increased compared to the Tₘₐₓ of the same anti-mast cell antibody administered intravenously. In certain embodiments, the anti-mast cell antibody is administered to the subject via a slow absorption delivery route such that the Tₘₐₓ of the anti-mast cell antibody is increased by at least 10%, at least 15%, at least 20%, at least 25%, or at least 30% compared to the Tₘₐₓ of the same anti-mast cell antibody administered intravenously.

In certain embodiments, the methods described herein reduce or prevent mast cell activation. In certain embodiments, the methods reduce or prevent mast cell degranulation. In certain embodiments, the methods reduce or prevent allergic reactions. In certain embodiments, the methods reduce or prevent anaphylaxis. In certain embodiments, the methods reduce the severity of allergic reactions. In certain embodiments, the methods reduce the severity of anaphylaxis.

In certain embodiments, administration of the anti-mast cell antibody does not trigger mast cell activation. In certain embodiments, administration of the anti-mast cell antibody does not trigger mast cell degranulation. In certain embodiments, administration of the anti-mast cell antibody does not trigger an allergic reaction. In certain embodiments, administration of the anti-mast cell antibody does not trigger anaphylaxis.

### Anti-Mast Cell Antibodies

The anti-mast cell antibodies administered according to the methods described herein are required to bind to/immunoreact with mast cells. More particularly, the anti-mast cell antibodies preferentially bind to a protein expressed on the surface of a mast cell, such as a receptor or transmembrane protein. Preferably, the target protein is uniquely expressed on mast cells, or is at least more abundantly expressed on mast cells than any other cell type. In this way, the methods of the invention can specifically target mast cells for depletion.

In certain embodiments, the anti-mast cell antibody specifically binds to a protein expressed on the surface of mast cells. In certain embodiments, the anti-mast cell antibody specifically binds to a mast-cell-specific surface protein. In certain embodiments, the anti-mast cell antibody specifically binds to a receptor expressed on the surface of mast cells. In certain embodiments, the anti-mast cell antibody specifically binds to a mast cell-specific receptor. In certain embodiments, the receptor is c-KIT. In certain embodiments, the anti-mast cell antibody is an anti-c-KIT antibody.

The term “antibody” is defined elsewhere herein and is used relatively broadly in the context of the present invention to refer to any antigen-binding molecule having binding specificity for a mast cell target. The term “antibody” as used herein is broad enough to encompass antigen-binding fragments as defined elsewhere herein. The term “antibody” also encompasses, but is not limited to, monoclonal antibodies (including full length monoclonal antibodies), polyclonal antibodies, multispecific antibodies (e.g., bispecific antibodies), VHH antibodies, so long as they exhibit the appropriate immunological specificity for a mast cell protein.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes) on the antigen, each monoclonal antibody is directed against a single determinant or epitope on the antigen.

“Antibody fragments” or “antigen binding fragments” comprise a portion of a full-length antibody, generally the antigen binding or variable domain thereof. Antibody fragments are described elsewhere herein and examples of antibody fragments include Fab, Fab′, F(ab′)2, bi-specific Fab's, and Fv fragments, diabodies, linear antibodies, single-chain antibody molecules, a single chain variable fragment (scFv) and multispecific antibodies formed from antibody fragments (see Holliger and Hudson, Nature Biotechnol. 23:1126-36 (2005), the contents of which are incorporated herein by reference).

The anti-mast cell antibodies for use in accordance with the methods described herein are intended for human therapeutic use and therefore, will typically be immunoglobulins of the IgA, IgD, IgE, IgG, IgM type, often of the IgG type, in which case they can belong to any of the four sub-classes IgG1, IgG2a and b, IgG3 or IgG4. In preferred embodiments, the anti-mast cell antibodies are IgG antibodies. Particularly preferred are IgG1 antibodies. Monoclonal antibodies are preferred since they are highly specific, being directed against a single antigenic site.

The anti-mast cell antibodies may exhibit high human homology. Such antibodies having high human homology may include antibodies comprising VH and VL domains of native non-human antibodies which exhibit sufficiently high % sequence identity to human germline sequences. In certain embodiments, the antibodies or antigen binding fragments thereof are humanised or germlined variants of non-human antibodies.

The Anti-Mast Cell Antibodies for Use in Accordance with the Methods Described Herein

May comprise or consist of one or more antibody variable domains connected to an Fc region, optionally via an antibody hinge region. In certain embodiments, the hinge region and/or Fc region have fully or substantially human amino acid sequence.

It is particularly preferred, in the context of the present invention, that the anti-mast cell antibody can participate in Fc-mediated effector functions in order to bring about efficient depletion of mast cells. The examples herein demonstrate that anti-mast cell antibodies possessing a functioning Fc region are able to deplete mast cells more effectively than anti-mast cell antibodies having an Fc-dead Fc region. Moreover, the data show that anti-mast cell antibodies with Fc regions that have been modified to enhance antibody-dependent cellular cytotoxicity (ADCC) activity perform particularly effectively with respect to mast cell depletion.

Accordingly, in certain embodiments, the anti-mast cell antibody comprises an Fc region possessing one or more Fc effector functions. In certain embodiments, the Fc region is modified to enhance one or more Fc effector functions. In certain embodiments, the Fc region possesses enhanced Fc effector function relative to a wild-type Fc region, particularly a wild-type human Fc region. In certain embodiments, the Fc region possesses enhanced Fc effector function relative to a wild-type human IgG Fc region, particularly a wild-type human IgG1 Fc region.

In certain embodiments, the one or more Fe effector functions are selected from: antibody-dependent cellular cytotoxicity (ADCC), antibody-dependent phagocytosis (ADCP), and complement dependent cytotoxicity (CDC). In certain preferred embodiments, the Fc region possesses or is modified to enhance antibody-dependent cellular cytotoxicity (ADCC).

In certain embodiments, the anti-mast cell antibody is a human IgG antibody. In certain embodiments, the anti-mast cell antibody is a human IgG1 antibody. In other embodiments, the anti-mast cell antibody is a human IgG2 antibody.

Anti-mast cell antibodies for use in accordance with the methods of the invention may be modified so as to enhance Fc effector function, particularly so as to enhance ADCC, via any suitable changes to the amino acid sequence and/or post-translational modifications known to those skilled in the art. Changes to the amino acid sequence of the Fc region so as to enhance Fc effector function may include amino acid substitutions, deletions and/or insertions. For instance, mutations may be introduced that improve binding affinity of Fc for specific FcγRs. In certain embodiments, the Fc region of the anti-mast cell antibody comprises the amino acid substitutions S239D/I332E.

Antibodies deficient in fucose have been shown to have 50-fold higher binding to FcγRIIIa and enhanced ADCC activity. During the upstream manufacturing stages, several strategies may be employed to control Fc glycosylation level including chemical inhibitors, culture process parameters, media supplementation, and cell line engineering. These strategies would be known to the skilled person. Accordingly, in certain embodiments, the Fc region of the anti-mast cell antibody is afucosylated. Terminal galactosylation (i.e. the glycosylation of galactose) of recombinant IgGs does not appear to affect the antibody binding to antigen, however it has been reported that the presence of galactose may result in increases in CDC activity of some IgGs. Accordingly, in certain embodiments, the Fc region of the anti-mast cell antibody is galactosylated.

In certain embodiments where the Fc region of the anti-mast cell antibody is modified to enhance one or more effector functions, the anti-mast cell antibody exhibits increased binding affinity for an FcγR compared to the unmodified antibody. In certain embodiments where the Fc region of the anti-mast cell antibody is modified to enhance one or more effector functions, the anti-mast cell antibody exhibits increased binding affinity for FcγRIIIa (CD16) compared to the unmodified antibody.

### Mast Cell Diseases

The present invention provides methods of treating a mast cell disease in a subject in need thereof. The present invention also provides for an anti-mast cell antibody for use in a method of treating a mast cell disease. In addition, the present invention provides for use of an anti-mast cell antibody in the manufacture of a medicament for treating a mast cell disease. In all instances, the therapeutic methods and uses comprise administering a therapeutically effective amount of an anti-mast cell antibody to the subject, wherein the anti-mast cell antibody is administered to the subject via a slow absorption delivery route.

The term “mast cell disease” is defined elsewhere herein. In certain embodiments, the mast cell disease to be treated in accordance with the therapeutic methods described herein is mastocytosis.

In certain embodiments of the invention, the subject is human. In certain embodiments, the subject has an abnormally high mast cell number. In certain embodiments, the subject has a high mast cell burden. It is usually possible to confirm mast cell numbers in the skin by carrying out a biopsy, where a small skin sample is taken and checked for mast cells. Other tests which can be used to confirm systemic mast cell numbers include measuring blood tryptase levels.

The anti-mast cell antibodies for use in accordance with the methods of the invention may be administered according to a suitable dosing regimen. It is particularly preferred that the anti-mast cell antibody is administered to the subject as is necessary to effectively deplete mast cell numbers. Accordingly, in certain embodiments, the method comprises administering one or more doses of the anti-mast cell antibody to the subject. In certain embodiments, the method comprises administering at least two doses of the anti-mast cell antibody. In certain embodiments, the method comprises administering at least three doses of the anti-mast cell antibody. In certain embodiments, the anti-mast cell antibody is administered to the subject daily.

In certain embodiments, the anti-mast cell antibody is administered to the subject once every 2 days. In certain embodiments, one or more doses of the anti-mast cell antibody is administered to the subject, wherein the one or more doses are administered to the subject once every 2 days. In certain embodiments, the anti-mast cell antibody is administered to the subject once every 7 days. In certain embodiments, one or more doses of the anti-mast cell antibody is administered to the subject, wherein the one or more doses are administered to the subject once every 7 days.

### Pharmaceutical Compositions

The anti-mast cell antibodies for use in the methods described herein may be formulated as pharmaceutical compositions for administration to the subject. Accordingly, in certain embodiments, the anti-mast cell antibody is formulated in a pharmaceutical composition.

Pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995. The term “pharmaceutically acceptable carrier” relates to carriers or excipients, which are inherently non-toxic. Examples of such excipients are, but are not limited to, saline, Ringer's solution, dextrose solution and Hanks' solution. Non-aqueous excipients such as fixed oils and ethyl oleate may also be used.

Pharmaceutical compositions typically must be sterile and stable under the conditions of manufacture and storage. The composition can be formulated as a solution, micro-emulsion, liposome, or other ordered structure suitable to high drug concentration. Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The pharmaceutical compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol, sorbic acid, and the like. It may also be desirable to include isotonicity agents, such as sugars, polyalcohols such as mannitol, sorbitol, glycerol or sodium chloride in the compositions. Pharmaceutically-acceptable antioxidants may also be included, for example (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmaceutical compositions comprising an anti-mast cell antibody must be administered to the subject in accordance with the presence invention via a slow absorption delivery route. For example, administration may be parenteral, preferably by subcutaneous or intradermal injection.

In addition to optimising the drug administration route, the pharmaceutical composition may be formulated to further slow the rate of drug absorption. Modified-release formulations are defined elsewhere herein and refer to pharmaceutical formulations whereby the timing and/or rate of drug release from the formulation is altered. In certain embodiments, the pharmaceutical composition is formulated for slow absorption of the anti-mast cell antibody. In certain embodiments, the pharmaceutical composition is a modified-release formulation. In certain embodiments, the pharmaceutical composition is a modified-release formulation selected from: slow-release, extended-release, prolonged-release, sustained-release, delayed-release or controlled-release.

The invention will now be further understood with reference to the following non-limiting examples.

### EXAMPLES

The following examples demonstrate the ability of anti-mast cell antibodies to achieve safe, effective mast cell depletion. The results demonstrate that anti-mast cell antibodies possessing Fc effector function can be administered without inducing anaphylaxis, in particular by subcutaneous injection. The anti-mast cell antibodies exemplified herein below, when administered subcutaneously, can achieve effective mast cell depletion and a clear reduction of mast cell-mediated inflammatory reactions. Importantly, antibody-mediated mast cell depletion successfully reduces severity of anaphylaxis, as demonstrated using a passive systemic anaphylaxis model.

### Example 1: Mast Cell Depletion with IgG Antibodies Targeting c-KIT

Mast cell are notoriously hard to target with antibodies due to the mast cells' ability to degranulate and induce anaphylaxis. The mouse IgG2a anti-c-KIT monoclonal antibody—clone ACK2—engineered so as to have enhanced ADCC activity (ADCC+) was found to induce hypothermia, a sign of anaphylaxis, in mice, after intraperitoneal injection.

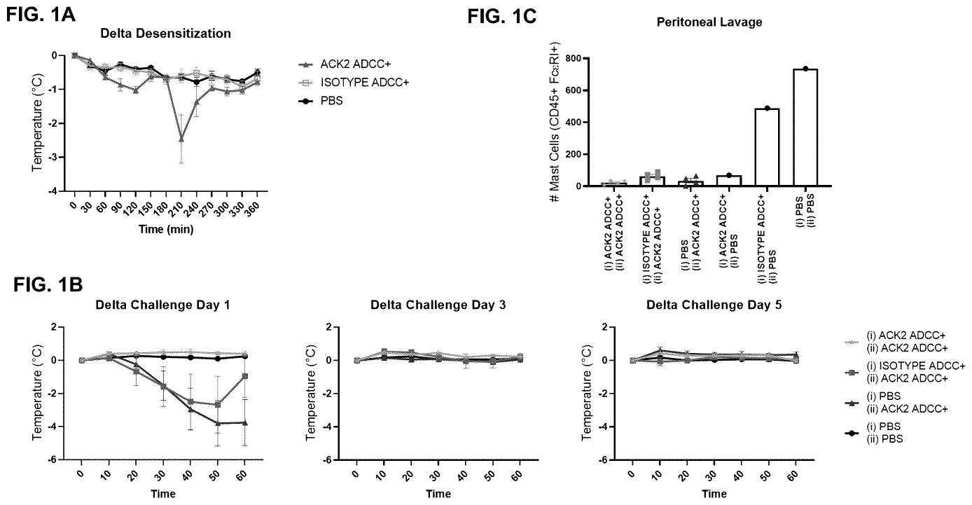

In a first experiment designed to address this issue, a “desensitization” approach was carried out, similar to what has been previously reported using an anti-FεRI antibody (Finkelman et al., 2020). It was hypothesized that mast cell activation could be prevented by dose escalation of the anti-c-KIT ADCC+ mAb. Mice received intraperitoneal injections of increasing doses of anti-c-KIT ADCC+ antibody, an isotype control or vehicle (PBS) at doses of 0.1, 0.3, 1, 3 and 10 μg/injection, every 30 minutes. The temperature of the mice was monitored, see FIG. 1A.

During the desensitization phase, the mice showed signs of anaphylactic shock once a dose of 3 μg was reached. This suggests that the repeated systemic antibody injections culminated in a dose that could activate the mast cells.

After the desensitization phase, the same mice were also challenged with a larger bolus of 30 μg of anti-c-KIT ADCC+ mAb (FIG. 1B, left panel (Day 1)). This desensitization with anti-c-KIT ADCC+ mAb seemed to work as there was no shock compared to the mice that has been administered isotype or PBS control during the desensitization phase, although the isotype seemed to have a reduced shock compared to the PBS control. This is likely due to blocking FcγRs. After a bolus dose, subsequent treatments did not induce a shock (FIG. 1B middle (Day 3) and right (Day 5) panels). This further suggested that this could be a safe approach. However, given that there was still a notable shock during the initial desensitization phase, there was a need to consider further approaches.

On day 7, mast cells were quantified in the different groups to determine the extent of depletion using this desensitization approach (FIG. 1C). Effective depletion of mast cells was seen in the peritoneal cavity. Interestingly, the same depletion was seen in all groups that received at least 3 injections of 30 μg of the anti-c-KIT ADCC+ mAb suggesting effective depletion is possible with this mAb. Given the positive indications from the desensitization approach, it was hypothesized that slower diffusion could be beneficial to improve the safety profile.

### Example 2: Effect of Different Routes of Administration of Anti-c-KIT Antibodies

The mouse IgG2a anti-c-KIT monoclonal antibody—clone ACK2 (which is commercially available)—was produced in different Fc backbone formats: Fc-dead (no effector function); wild-type; and ADCC+ (modified so as to possess enhanced ADCC function). The ADCC+ format contains the amino acid substitutions S239D/I332E in the Fc region.

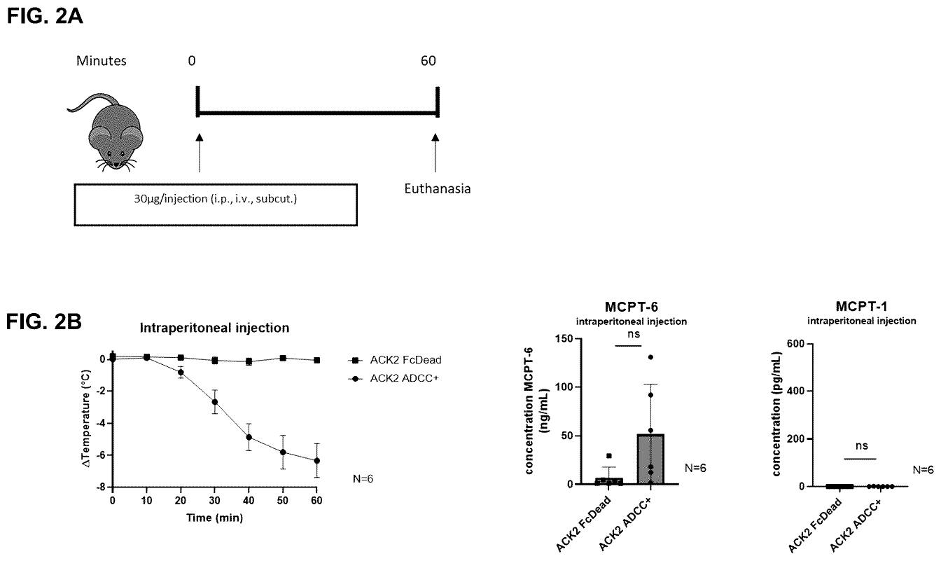

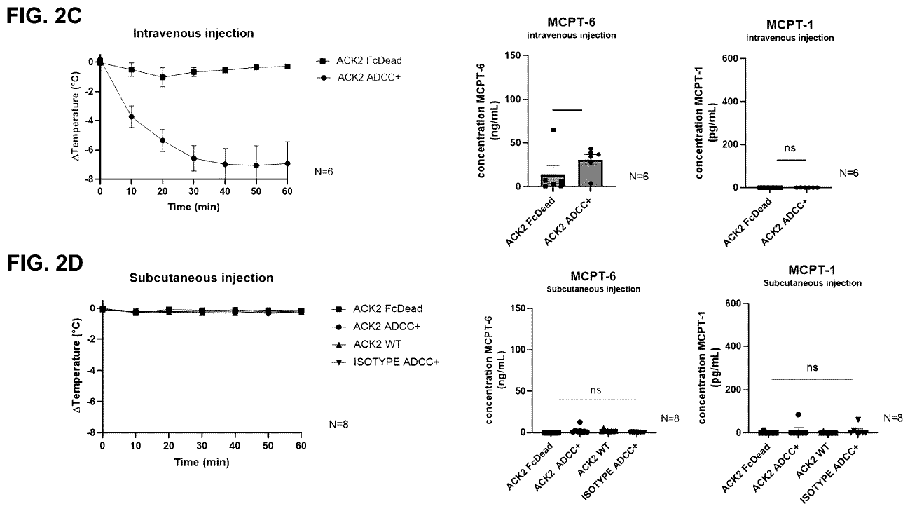

As shown in FIG. 2, wild-type mice were injected with 30 μg of either Fc-dead or ADCC+ mAb intraperitoneally (FIG. 2B), intravenously (FIG. 2C), or subcutaneously (FIG. 2D). The subcutaneous experiments also included wild-type ACK2 and Isotype control.

In no circumstance did the Fc-dead mAb induce hypothermia. The results show that intravenous injection of the anti-c-KIT ADCC+ mAb induced the most rapid anaphylaxis, with intraperitoneal injection showing a slightly delayed but similar reaction. Most interestingly, subcutaneous injection did not induce any hypothermia at all with any antibody: isotype control, anti-c-KIT Fc-dead, WT or ADCC+. This is likely due to the slower diffusion of the antibodies through the subcutaneous tissue.

One hour after the challenge with the antibodies, blood samples were taken and serum isolated, which was subsequently used to quantify the release of mast cell-specific proteases MCPT-1 (produced by mucosal mast cells) and MCPT-6 (produced by connective tissue-type mast cells). In line with the hypothermia results, the data show that MCPT-6 was released at high levels when the anti-c-KIT ADCC+ was injected i.v. or i.p., but not with the Fc-dead antibody. Wild-type and anti-c-KIT ADCC+ antibodies injected subcutaneously had no or very slightly elevated levels of MCPT-6; however, not in the same range at all to the other routes of injection. Isotype control and anti-c-KIT Fc-dead antibody-treated groups had no detectable MCPT-6. MCPT-1 levels were undetectable in almost all samples regardless of the route of injection, indicating that mucosal mast cells (the only mast cells that express MPCT-1) are not activated by this treatment. These data confirm a solid safety profile for anti-c-KIT antibodies having Fc effector function when injected subcutaneously.

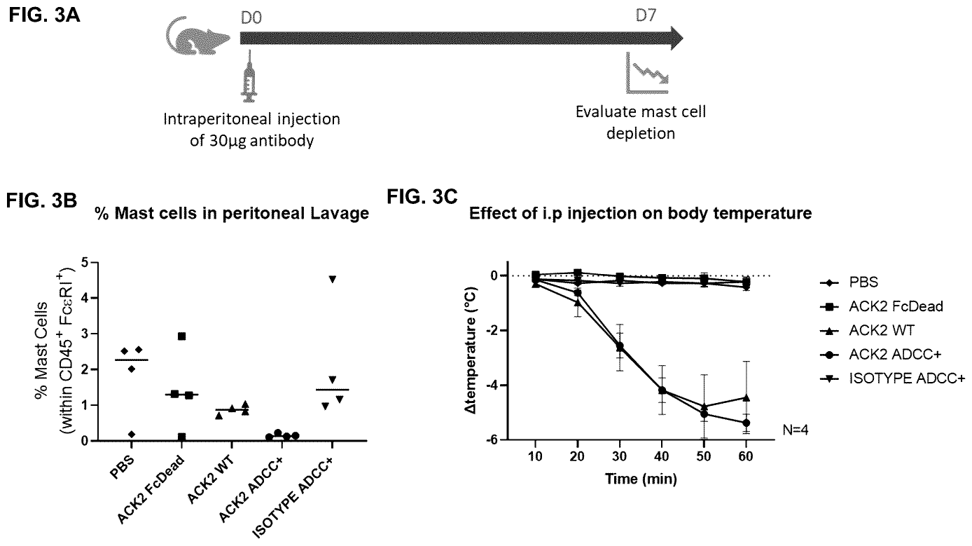

FIG. 3 shows that rapid onset of anaphylaxis occurs following i.p. injection of 30 μg anti-c-KIT wildtype and anti-c-KIT ADCC+ (FIG. 3C). The percentage of mast cells present in the peritoneal cavity was also assessed on day 7 following administration. The data shows that all formats of the anti-c-KIT antibody are capable of reducing mast cell numbers when administered intraperitoneally, with anti-c-KIT ADCC+ being most effective, followed by anti-c-KIT wild-type, followed by anti-c-KIT Fc-dead (FIG. 3B).

### Example 3: Subcutaneous Anti-c-KIT Antibody (ACK2 ADCC+) Injection can Deplete Mast Cells without Triggering Anaphylaxis

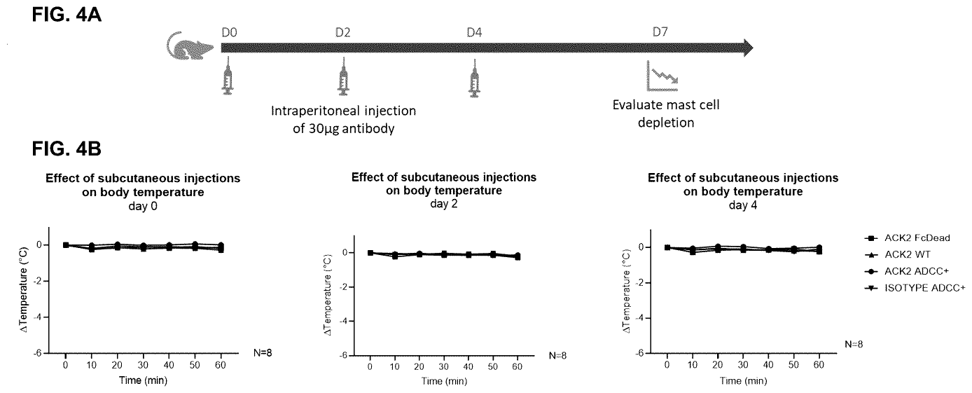

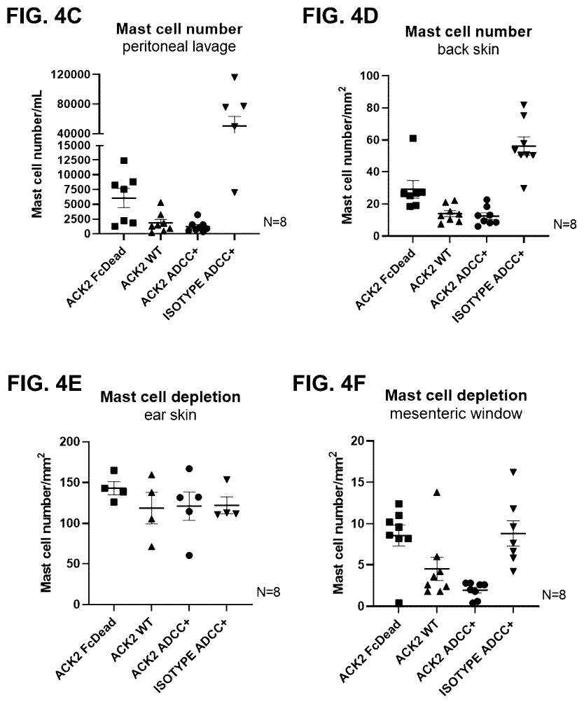

In Example 3, C57BL/6 wild-type mice were administered three anti-c-KIT antibody injections subcutaneously over one week (on Days 0, 2 and 4) followed by mast cell quantification on day 7 (FIG. 4A). As shown in FIG. 4B, the anti-c-KIT antibodies do not induce anaphylaxis when administered subcutaneously, regardless of the antibody format. This 3-dose administration schedule resulted in effective depletion of mast cells in the peritoneal cavity with all three anti-c-KIT antibody formats (Fc-dead, wild-type, and ADCC+) compared to control, with the most pronounced reduction observed for the ADCC+ antibody (see FIG. 4C). The same trend in mast cell quantification was observed in back skin and mesenteric windows (see FIGS. 4D and 4F). No significant mast cell depletion was observed in the ear skin (see FIG. 4E); however, this is not surprising as the ears have a large mast cell burden in mice (approximately 3 times that of the back skin) and depletion in this tissue would likely take a much longer treatment protocol.

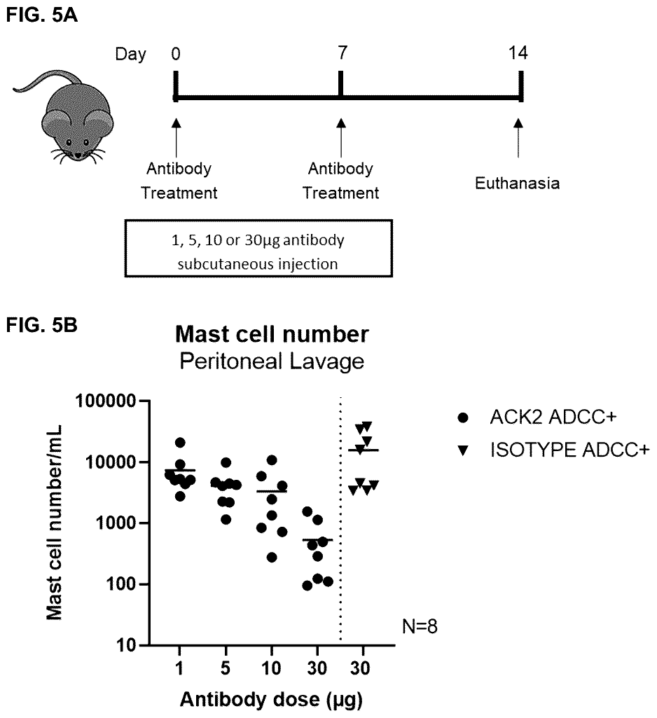

An alternative treatment protocol was investigated, which involved two subcutaneous injections one week apart and allowing for a longer time period in which to deplete the mast cells (FIG. 5A). As can be seen in FIG. 5B, 30 μg was the optimum dose depleting approximately 90% of mast cells in the peritoneal cavity.

### Example 4: Efficacy of Subcutaneous Injections of Anti-c-KIT Antibodies on Mast Cell Number in Different Organs

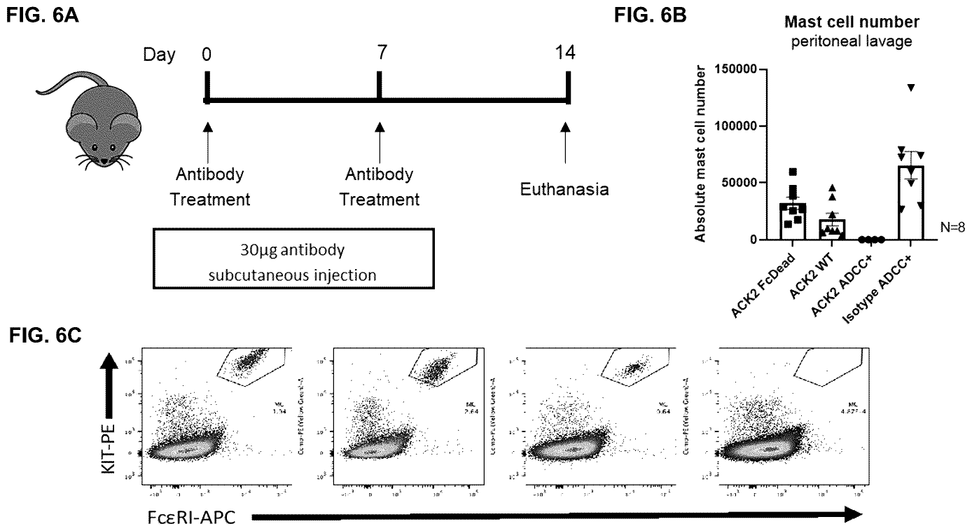

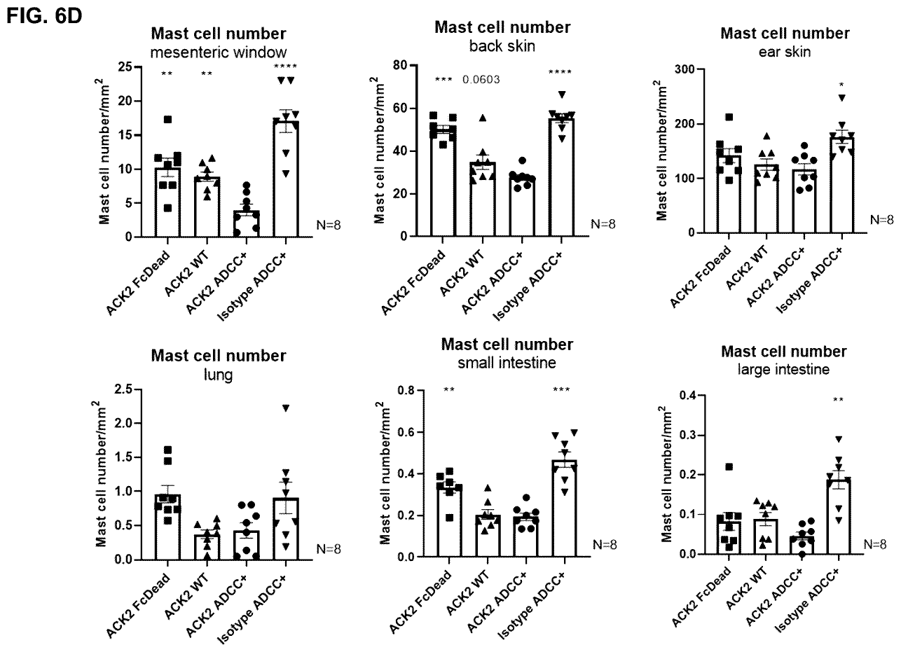

The different anti-c-KIT antibody formats were tested side by side following the protocol depicted in FIG. 6A and mast cell numbers in various tissues were analysed. FIGS. 6B and 6C show the mast cell profile in the peritoneal cavity. Significantly more depletion of absolute mast cell numbers was observed following administration with the anti-c-KIT ADCC+ antibody compared to the Isotype control and the Fc-dead-treated groups.

Histological analysis was also performed as shown in FIG. 6D. Mesenteric windows were stained with Csaba Stain, and results show that the ADCC+ format was significantly more effective in depletion than the isotype control and the other formats of anti-c-KIT mAbs. Back skin and ear skin were stained with toluidine blue, and this revealed that the ADCC+ format continued to be effective at depletion compared to the isotype control in both tissue samples. The anti-c-KIT ADCC+ antibody was also more effective as compared to the Fc-dead format in the back skin section, and is trending towards significance in the ear skin when compared to the WT format treatment (p=0.0603). Lung sections were also analysed by toluidine blue staining; however, this only stains heparin positive mast cells around the bronchi. This gives rise to variability in mast cell numbers based on the section analysed. Finally, the small and large intestine sections were analysed for mast cells with MCPT-1 immunofluorescent staining. This also clearly showed that mast cells were significantly depleted by anti-c-KIT ADCC+ antibody in both intestinal tissues compared to the control group. In the WT treated group, mast cells were also depleted in small intestine but less significantly than the ADCC+ antibody treated group.

### Example 5: Effects of Anti-c-KIT mAbs on Leukocyte Populations and Bone Marrow Progenitors

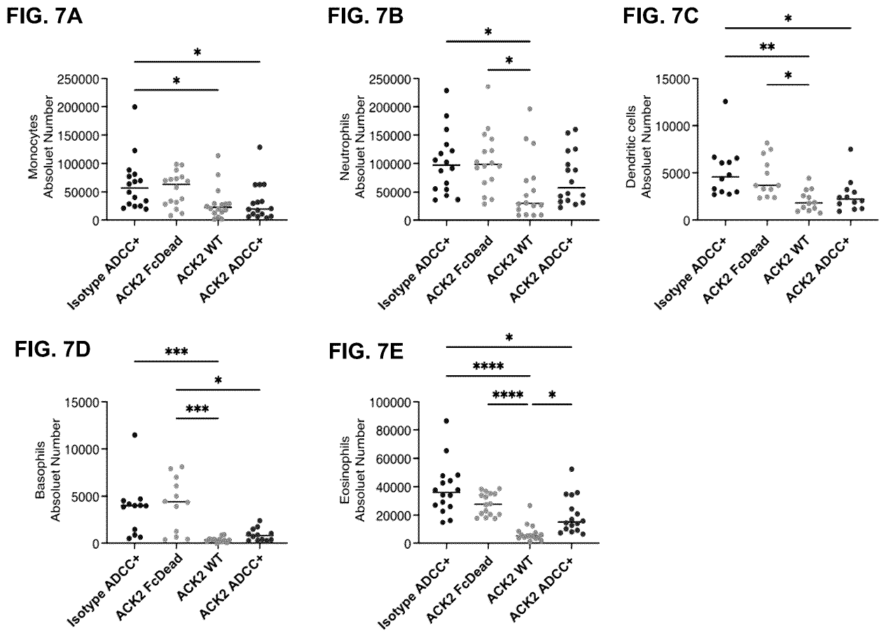

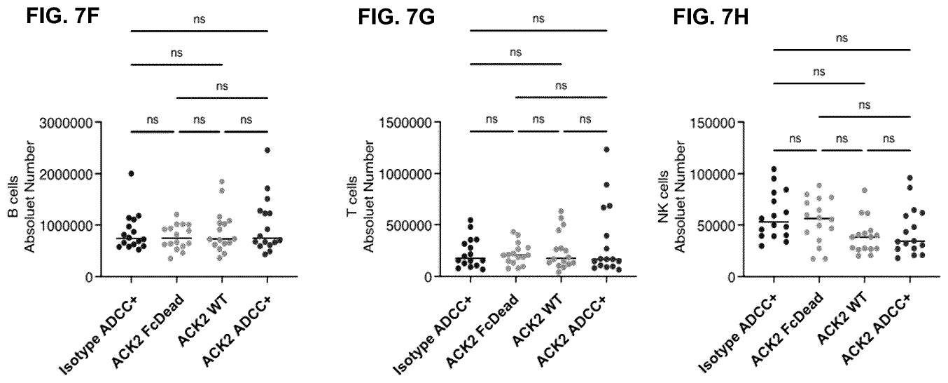

Given that the common progenitor of many leukocytes expresses the c-KIT receptor, a full leukocyte panel was run to examine the effect of treatment on the major immune populations in the blood (FIG. 7). This is an essential safety aspect of targeting c-KIT, as often treatments in the clinic, specifically Tyrosine Kinase Inhibitors (TKIs), result in immunosuppression due to the non-specific inhibition of tyrosine kinase receptors globally.

Blood was sampled on day 13 (one day before sacrifice) and quantitative FACS was carried out on 200 μl of blood using counting beads. No major variations in the absolute numbers of the major lymphocytes (B cell, T cell, and NK cells) were observed (see FIGS. 7F, 7G and 7H, respectively). This indicates that a competent adaptive immune system is still present regardless of the antibody format. However, when examining cell populations from myeloid origins, significant changes were observed following treatment with WT and ADCC+ anti-cKIT mAbs, but not with the Fc-dead format (see FIGS. 7A-E). WT treatment resulted in a significant reduction in the following myeloid populations: monocytes, neutrophils, dendritic cells, basophils and eosinophils. Similarly, ADCC+ antibody treatment resulted in reductions in these same myeloid cell populations, with the exception of the neutrophil population which was reduced but not to a significant degree. These reductions therefore appear to be dependent on the Fc effector function.

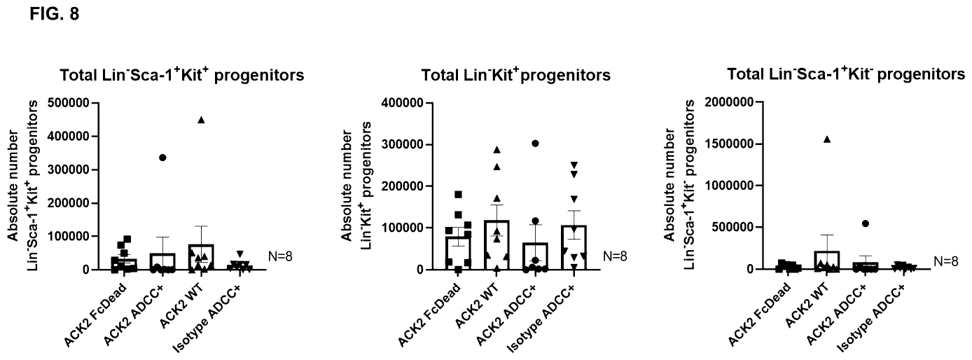

Because bone marrow progenitors also express the c-KIT receptor, progenitor populations at day 14 of the depletion protocol were examined. Following the same treatment protocol as shown in FIG. 6A, the levels of Lin⁻Sca-1⁺Kit⁺ (LSK), Lin⁻Kit⁺ (LK), and Lin⁻ Sca-1⁺Kit⁻ (LSK−) cells from the bone marrow were measured (FIG. 8). No significant difference was observed between controls and all treatments groups. This is indicative of a good safety profile as it means there will likely be less immunosuppressive effects, especially compared to current treatments, such as imatinib or avapritinib, which are small molecule TKIs that inhibit c-KIT but also other receptors. These results also suggest that the effects observed in the blood myeloid analysis is more likely due to tissue recruitment or potentially depletion of circulatory progenitor cells.

### Example 6: Efficacy of Mast Cell Depletion in Reducing Anaphylaxis

To observe the effects of anti-c-KIT antibody treatment on mast cell-mediated reactions, two different IgE-induced passive systemic anaphylaxis protocols were carried out following the administration of mast cell depleting antibodies.

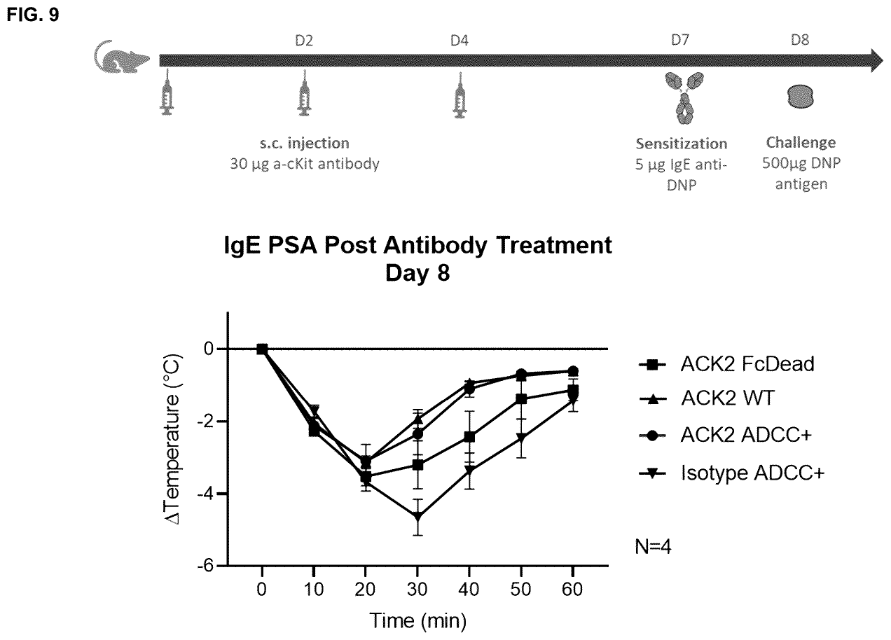

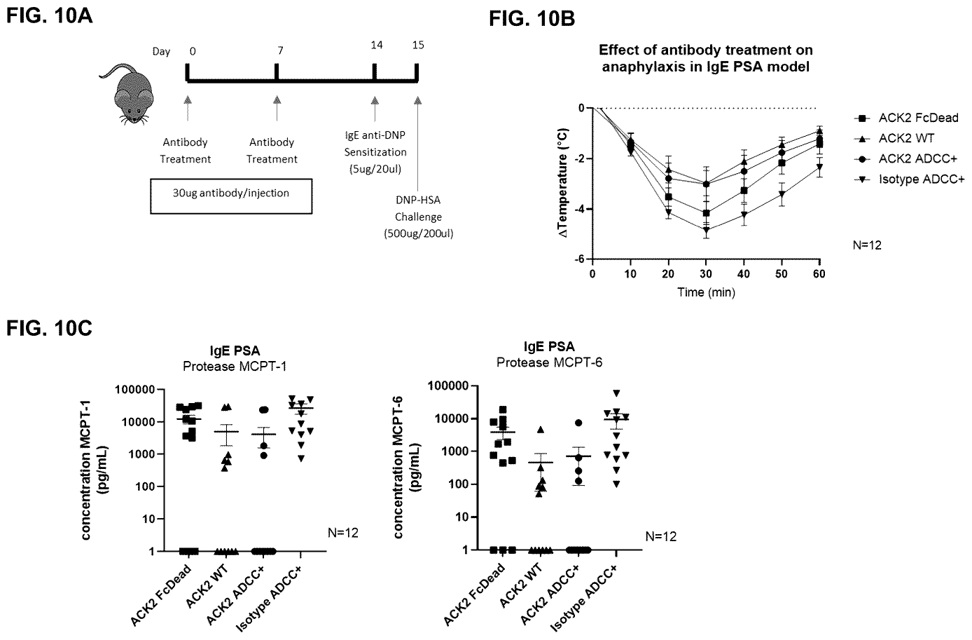

In one study, mast cell depletion was carried out with IgE sensitisation on day 7 and antigen challenge on day 8, as shown in FIG. 9. In a second study, mast cell depletion was carried out with IgE sensitisation on day 14 and antigen challenge on day 15, as shown in FIG. 10A.

In both studies, core body temperature was monitored for an hour after the challenge to examine hypothermia, the golden standard of anaphylaxis in mice. The control mice had a clear drop in temperature averaging around 5 °C. Fc-dead anti-c-KIT antibody treated mice seemed to be slightly protected from this temperature drop. However, in the WT and ADCC+ anti-c-KIT antibody treated groups, the hypothermia was markedly less severe (see FIG. 9 and FIG. 10B). Although clinical scoring was not carried out, clear behavioural differences were observed with WT and ADCC+ antibody treated mice acting normally after challenge, and Isotype and Fc-dead groups exhibiting scratching, malaise and prostrate positioning.

In the second study, at 60 minutes post-challenge, blood was harvested for serum analysis of mast cell specific proteases, MCPT-1 and MCPT-6, which were later quantified by ELISA (FIG. 10C). This serum analysis revealed that the ADCC+ format most significantly reduced serum MCPT-1 compared to the isotype control, although the WT treated group also significantly reduced the levels of MCPT-1. The same trend was observed when examining MCPT-6 levels where both WT and ADCC+ treated groups had significantly reduced levels of this protease in the serum. Both MCPT-1 and MCPT-6 were reduced 10-fold on average in WT and ADCC+ treated groups, and in some samples were completely undetectable. In the Fc-dead treated group some samples also had undetectable levels of proteases; however, in most samples the levels remained at similar levels to the isotype control group.

What is most promising is that ADCC+ antibody treatment reduced MCPT-1 significantly compared to the Fc-dead treated group. This further shows that an ADCC+ antibody format is better than the Fc-dead format at depleting mast cells, thereby also providing better protection against IgE-mediated anaphylaxis and provides further evidence that for aggressive mast cell mediated diseases, antibodies without Fc effector function (e.g. barzolvolimab) would be less efficient.

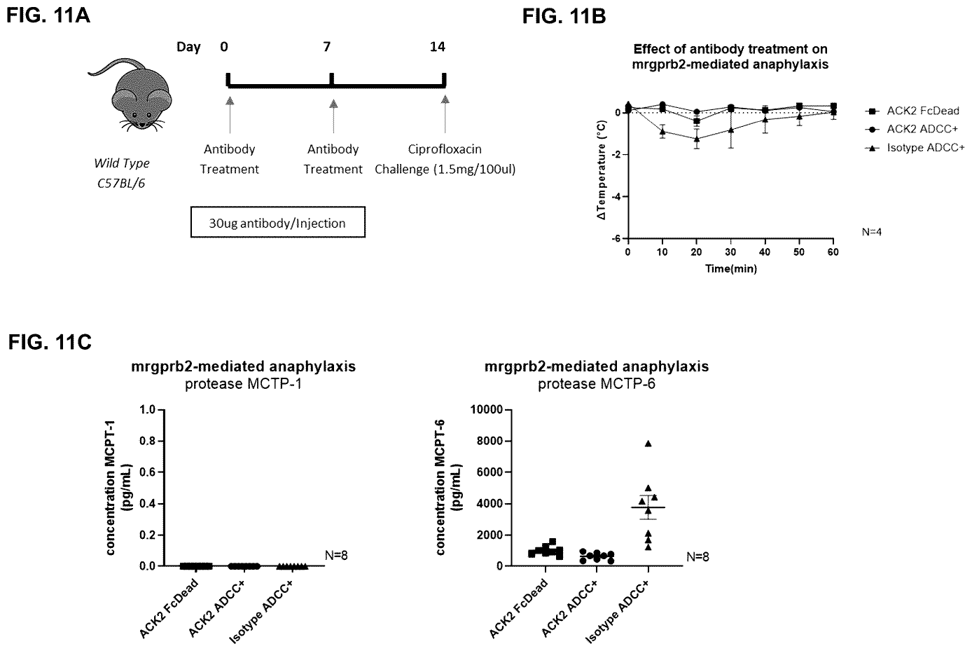

To further test the protective effects of mast cell depletion, another mast cell-dependent reaction was carried out. In this further study, ciprofloxacin-induced passive systemic anaphylaxis was assessed (FIG. 11A). Ciprofloxacin-induced passive systemic anaphylaxis is known to be a mast cell and mrgprb2 (the mouse ortholog of MRGPRX2) dependent reaction, since mrgprb2 KO mice are protected from signs of anaphylaxis in this PSA model. Mast cell activation by engagement of mrgprb2 is known as a pseudo-allergic reaction i.e., not an IgE-induced reaction. It is markedly less severe in terms of hypothermia, as evidenced by the ~1.5 °C temperature loss in the control (FIG. 11B). This experiment showed that the Fc-dead treatment resulted in significantly less severe hypothermia, and the ADCC+ treatment provided complete protection from hypothermia.

Serum was collected 60 mins post-challenge, and MCPT-1 and MCPT-6 ELISAs were carried out as markers of mast cell activation (FIG. PC). MCPT-1 could not be detected in any of the treated groups. This is due to the ciprofloxacin only activating mast cells that express the mrgprb2 receptor, which is restricted to connective tissue mast cells that don't express MCPT-1. However, MCPT-6 release was observed in the Isotype treated group. Both Fc-dead and ADCC+ antibody treated groups had significantly reduced serum MCPT-6, and more promising is that the MCPT-6 levels were even more significantly reduced with the ADCC+ treatment.

### Materials & Methods

TABLE 5
Antibodies

| | | | | |
|---|---|---|---|---|
| Target | Clone | Color | Reference | Provider |

In Vitro/Ex Vivo

| | | | | |
|---|---|---|---|---|
| GR-1 | RB6/6B2 | FITC | 11 - 9668. | eBioScience |
| mCD115 | REA827 | PE-Vio770 | 130-112-641 | Miltenyi |
| mCD117 (mc- | ACK2 | APC/BV421 | 135108 | Biolegend |
| Kit) | | | | |
| mCD117 (mc- | 2B8 | APC/PE | 105827 | Biolegend |
| Kit) | | | | |
| mCD11b | M1/70 | eFluoro450/FITC | 48-0112-82 | eBioscience |
| mCD131 | JORO50 | PE | 559920 | BD |
| mCD150 | TC15-12F12.2 | PE-Cy7 | 60036CZ.1 | StemCell |
| | | | | Tech. |
| mCD19 | 1D3 | BV421 | 562701 | BD |
| mCD3 | 17A2 | BV421 | 564008 | BD |
| mCD3 | 145-2C11 | FITC | 11-0031 | eBioscience |
| mCD45 | 30-F11 | APC-Cy7 | 557659 | BD |
| mCD45Ra | RA3/6B2 | FITC | 11-0452 | eBioscience |
| mCD48 | HM48-1 | APC | 17-0481 | eBioscience |
| mCD49b | HMα2 | FITC | 103503 | Biolegend |
| mD11c | HL3 | PE-CF594 | 562454 | BD |
| mF4/80 | REA126 | APC | 130-116-525 | Miltenyi |
| mFcεRI | REA1079 | APC | 130-118-839 | Miltenyi |
| mLy6C | AL-21 | BV605 | 563011 | BD |
| mLy6G | A18 | FITC | 551460 | BD |
| mMHC II (I- | M5/114.15.2 | AF700 | 107622 | BD |
| A/I-E) | | | | |
| mMPCT-1 | RF6.1 | n/a | 14-5503-82 | eBioscience ™ |
| mNK1.1 | PK136 | BV786 | 568224 | BD |
| Propidium | n/a (live/dead) | n/a | 130-093-233 | Miltenyi |
| Iodide | | | | |
| Rat IgG | Goat IgG | AF657 | A-21247 | ThermoFisher |
| mSca-1 | E13-161.7 | Biotin | 553334 | BD |
| mSiglec F | E50-24400 | BV711 | 552126 | BD |
| Streptavidin | n/a | BV421 | 405225 | Biolegend |
| Sytox Blue | n/a (live/dead) | (BV510) | S11350 | Thermofisher |
| mTer119 | Ter119 | FITC | TNB35-5921 | Ozyme |
| TruStain FcX | 93 | n/a | 101320 | Biolegend |
| (Fc Block) | | | | |

In Vivo

| | | | | |
|---|---|---|---|---|
| mouse c-Kit | ACK2 (WT) | n/a | n/a | Argenx |
| mouse c-Kit | ACK2 (Fc | n/a | n/a | Argenx |
| | Dead) | | | |
| mouse c-Kit | ACK2 | n/a | n/a | Argenx |
| | (ADCC+) | | | |
| RSV | Motavizumab | n/a | n/a | Argenx |
| glycoprotein F | (ADCC+) | | | |
| DNP | SPE-7 (IgE) | n/a | D8406 | SIGMA |

### Mice

Wildtype 7-week-old C57BL/6 mice were obtained from Charles River Laboratories, and were cared for by the CREFRE animal facility (Toulouse, Purpan). Mice were utilised from 8 weeks of age, unless otherwise specified. Local ethics committee approval was acquired for all experiments.

### Anti-c-Kit mAb Treatment

30 μg/100 μl of anti-mouse c-KIT (CD117) mAb (clone ACK2) in different formats (Fc-dead, WT or ADCC+) or Isotype control (ADCC+) were injected either subcutaneously (s.c), intraperitoneally (i.p) or intravenously (i.v), when testing the safety profile. In all other experiments, 30 μg/100 μl of these antibodies were injected subcutaneously, unless otherwise specified.

The ACK2 monoclonal antibody reacts with mouse CD117, also known as the c-KIT receptor. ACK2 has been reported to block c-KIT function. Motavizumab is an antibody against respiratory syncytial virus (RSV) and was used as an isotype control.

### IgE Passive Systemic Anaphylaxis (PSA)

Mice were sensitized with 10 μg/200 μl IgE anti-DNP (clone SPE-7). 24 hours later mice were challenged with intravenous injection of 500 μg/200 μl DNP-I (Sigma). Anaphylaxis was observed as a decrease in body temperature (rectal measurement) for one hour.

### Tissue Samples

Tissue samples (back skin, ear skin, lung, small and big intestine) were harvested and placed in formaldehyde (4% w/v) for 24 hours at room temperature. After this, samples were placed in 70% EtOH at 4 °C until included in paraffin blocks. Sections were cut at a thickness of 5 μm.

### Toluidine Blue

Briefly, sections were deparaffinated using xylene and decreasing concentrations of ethanol. Subsequently, sections were stained with toluidine blue (Sigma) for 30 seconds followed by rinsing in deionised water. Slides were mounted and scanned using a slide scanner (Panoramic 250). Mast cells were quantified using case viewer and represented as mast cell number/mm².

### Csaba Staining

Mesenteric windows were harvested directly onto Superfrost™Plus adhesion microscope slides, allowed to dry and then fixed in Carnoy fixative (3 EtOH: 2 Chloroform: 1 Acetic Acid). Excess tissue was removed (e.g., intestinal tissue/adipose tissue) and samples were stained in Csaba Staining for 15 mins at r.t. and subsequently washed in deionised water and then dehydrated using xylene. Slides were mounted and imaged quantified as number of mast cells/mm².

### Immunofluorescence (MCPT-1 Staining)

Briefly, sections were deparaffinated using xylene and decreasing concentrations of ethanol. Subsequently, sections underwent antigen recover protocol in citrate buffer at 95 °C for 20 mins and allowed to cool in this buffer for 20-30 mins. Next, sections were blocked using 1% BSA PBS for 2 hours at r.t. after which sections were washed with PBS and stained with primary rat anti-mMCPT1 (clone RF6.1) (final concentration: 2.5 ug/ml) overnight at 4 °C. The next day goat anti-rat secondary antibody (AF657) was used to stain the primary antibodies for 2 hours at r.t. Slides were washed and mounted with Dap-prolong gold to allow visualisation of the entire tissue section. Images were acquired using the Zeiss confocal microscope and or by scanning with a slide scanner (panoramic 250).

### Serum Collection and ELISAs

Blood was collected at endpoint. Blood was allowed to coagulate at room temp for 30 minutes and subsequently samples were spun down at 10,000×g for 10 minutes. Serum was collected and stored at −20 °C until analysed.

MCPT-1 ELISA was carried out according to the manufacturer's instruction (Thermofisher, BMS6005). 1 in 50 dilution of serum in assay buffer was used.

MCPT-6 ELISA was carried out according to the manufacturer's instructions (Thermofisher, #EM51RB). 1 in 2 dilution of serum in assay buffer was used.

### Flow Cytometry

Flow cytometry acquisition was carried out on MACQuant10 (Miltenyi) or on FORTESSA (BD Bioscience). Analysis was carried out using FlowJo (TreeStar).

### Blood

Blood samples were harvested in 5 μM EDTA PBS (ice cold) containing counting beads. Samples were centrifuged (300 g, 5 mins) and 1 mL of ACK buffer (Gibco) was used to lyse red blood cells three times, washing with 1 mL PBS and centrifuging (300 g, 5 mins) in between each ACK lysis. Cells were blocked using TruStain (also known as Fc Block or anti-mCD32/16) for 10 minutes. Cells were washed with FACS buffer and then stained with antibody mix containing markers for major leukocyte population markers: Monocyte (CD11b+, Ly6C+, Ly6G−), Monocyte-derived Dendritic Cells (CD45+, CD11b+, CD11c+, MHCII+), Neutrophils (CD11b+, Ly6G+), Eosinophils (SSChi, CD11b+, Siglec F+), Basophils (CD45lo, CD131+), NK Cells (CD45+, NK1.1+), T Cells (CD45+, CD3+, MHCII−), and B Cells (CD45+, CD19+, MHCII+).

### Peritoneal Lavage

Peritoneal lavage was harvested using ice-cold 5 μM EDTA PBS containing counting beads. Samples were centrifuged and blocked with TruStain (as above). Next, cells were washed and stained for mast cells (CD45+, FεRI+ and c-Kit+ (clone 2B8)).

### Bone Marrow

One whole femur was harvested and kept in ice-cold 5 μM EDTA PBS. Bones were crushed using a mortar and pestle with the EDTA-PBS Solution containing counting beads. Total liquid was recovered and passed through a 70 μm cell strained to remove bone and other tissue debris. Sample were centrifuged at (300 g, 5 mins). Supernatant removed and red blood cell lyses carried out (as above). Cells were then stained using antibody mix: lineage (CD11b+, B220+, Ter119+, CD3+, Ly6G+, Ly6C+), cKIT, Sca-1 (Ly6A), CD34, IL-7Ralpha, CD16/32. Lineage-Kit+ (LK) cells and Lineage-Sca-1+Kit+ (LSK) were quantified as potentially affected by the different treatment. cKIT expression was assessed using the antibody clone 2B8 which does not compete with the clone ACK2. Lineage-Sca-1+Kit− cells were used as an internal control as these cells should not be directly affected by any anti-cKIT antibody treatment.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

Various publications and patent applications are cited herein, the disclosures of which are incorporated by reference in their entireties.

1. A method of depleting mast cells in a subject, the method comprising administering an effective amount of an anti-mast cell antibody to the subject, wherein the anti-mast cell antibody is administered to the subject via a slow absorption delivery route.

2. A method of treating a mast cell disease in a subject in need thereof, the method comprising administering a therapeutically effective amount of an anti-mast cell antibody to the subject, wherein the anti-mast cell antibody is administered to the subject via a slow absorption delivery route.

3. The method of claim 1 or claim 2, wherein the anti-mast cell antibody is administered subcutaneously or intradermally.

4. The method of claim 3, wherein the anti-mast cell antibody is administered subcutaneously.

5. The method of claim 4, wherein the anti-mast cell antibody is administered via a subcutaneous implant.

6. The method of claim 1 or claim 2, wherein the anti-mast cell antibody is not administered intravenously.

7. The method of any one of the preceding claims, wherein the anti-mast cell antibody specifically binds to a protein expressed on the surface of mast cells.

8. The method of any one of the preceding claims, wherein the anti-mast cell antibody is an anti-c-KIT antibody.

9. The method of any one of the preceding claims, wherein the anti-mast cell antibody comprises an Fc region.

10. The method of claim 9, wherein the Fc region possesses one or more Fc effector functions.

11. The method of claim 9, wherein the Fc region possesses one or more enhanced Fc effector functions relative to a wild-type Fc region.

12. The method of claim 10 or claim 11, wherein the one or more Fc effector functions are selected from: antibody-dependent cellular cytotoxicity (ADCC); antibody-dependent phagocytosis (ADCP); and complement dependent cytotoxicity (CDC).

13. The method of claim 12, wherein the Fc region possesses the Fc effector function antibody-dependent cellular cytotoxicity (ADCC).

14. The method of claim 12, wherein the Fc region possesses enhanced Fc effector function antibody-dependent cellular cytotoxicity (ADCC) relative to a wild-type Fc region.

15. The method of any one of the preceding claims, wherein the anti-mast cell antibody is a human IgG antibody.

16. The method of claim 15, wherein the Fc region comprises the amino acid substitutions S239D/I332E.

17. The method of any one of claims 9-16, wherein the Fc region is afucosylated.

18. The method of any one of claims 9-17, wherein the Fc region is galactosylated.

19. The method of any one of claims 2-18, wherein the mast cell disease is mastocytosis.

20. The method of any one of the preceding claims, wherein the subject has an abnormally high mast cell number.

21. The method of any one of the preceding claims, wherein the subject is a human.

22. The method of any one of the preceding claims, wherein the anti-mast cell antibody is formulated in a pharmaceutical composition.

23. The method of claim 22, wherein the pharmaceutical composition is formulated for slow absorption of the anti-mast cell antibody.

24. The method of claim 23, wherein the pharmaceutical composition is a modified-release formulation selected from: slow-release, extended-release, prolonged-release, sustained-release, delayed-release, or controlled-release.

25. The method of any one of the preceding claims, wherein the method reduces or prevents mast cell activation or mast cell degranulation.

26. The method of any one of the preceding claims, wherein the method reduces or prevents allergic reactions or anaphylaxis.

27. The method of any one of the preceding claims, wherein administration of the anti-mast cell antibody does not trigger mast cell degranulation, an allergic reaction and/or anaphylaxis.

28. An anti-mast cell antibody for use in treating a mast cell disease according to the method of any one of claims 1-27.

29. Use of an anti-mast cell antibody in the manufacture of a medicament for treating a mast cell disease wherein the treatment is carried out according to the method of any one of claims 1-27.
